(19) United States
(12) Patent Application Publication
(10) Pub. No.: US 2025/0135126 A1
(43) Pub. Date: May 1, 2025

(54) ELECTRICAL SYSTEM FOR A DRUG DELIVERY DEVICE AND DRUG DELIVERY DEVICE

(71) Applicant: Sanofi, Paris (FR)

(72) Inventor: Paul Richard Draper, Warwick, Warwickshire (GB)

(21) Appl. No.: 18/837,370

(22) PCT Filed: Feb. 9, 2023
(86) PCT No.: PCT/EP2023/053181

§ 371 Date: Aug. 9, 2024

(30) Foreign Application Priority Data
Feb. 11, 2022 (EP) 22315032.7

(51) Int. Cl. A61M 5/50

(52) U.S. Cl. CPC A61M 5/5086

(57) ABSTRACT

An electrical system for a drug delivery device is proposed, the electrical system comprising an electrical liquid sensitive unit, wherein the electrical liquid sensitive unit comprises at least one liquid sensing arrangement, wherein the electrical liquid sensitive unit is configured to react to the exposure of the liquid sensing arrangement to liquid, and wherein the electrical system is configured to change its state from a normal state to an error state in response to the exposure of the at least one liquid sensing arrangement to liquid.

### CROSS-REFERENCE TO RELATED APPLICATIONS

This is a National Stage Application, filed under 35 U.S.C. § 371, of International Patent Application No. PCT/EP2023/053181, filed on Feb. 9, 2023, which claims priority to European Patent Application No. 22315032.7, filed on Feb. 11, 2022, the disclosures of which are incorporated herein by reference in their entireties.

### TECHNICAL FIELD

The present disclosure relates to an electrical system for a drug delivery device. The disclosure further relates to a drug delivery device, for example a device comprising the electrical system.

### BACKGROUND

Drug delivery devices including or configured to be operated with one or more electronic components are becoming increasingly popular within the industry and also with users as they offer a variety of advantages over conventional drug delivery devices which do not use electronics but rely on mechanical systems only. An example for such a drug delivery device is disclosed in WO 2019/101962 A1. Here, a sensor system is used to determine the size of the delivered dose based on optically conducted measurements of the rotation of one member of the dose setting and drive mechanism of the drug delivery device relative to another member of that mechanism during the dose delivery operation. The determined doses can be used to generate a dose log on the doses the user has delivered from the device. Devices with electronic components are, of course, more sensible with respect to external influences acting on the electronic components, such as exposure to liquid, as compared to devices without electronics.

### SUMMARY

It is an object of the present disclosure to provide a novel, preferably an improved, electrical system for a drug delivery device and a device comprising such a system.

This object is, for example, achieved by the subject-matter of the independent claim, where the present disclosure should not be understood as being restricted to the subject-matter claimed therein that claim. Further expediencies, advantages, and refinements of the disclosure are set forth below and in the dependent claims.

One aspect of the present disclosure relates to an electrical system for a drug delivery device. Another aspect of the present disclosure relates to a drug delivery device comprising the electrical system. Therefore, the features which are disclosed herein with respect to the electrical system are also disclosed with respect to the device and vice versa.

In one embodiment, the electrical system comprises at least one electrical unit. The electrical unit may be an electronic unit, e.g. comprising at least one electronic component, or a non-electronic unit, e.g. an electrical power supply. The system may comprise a plurality of electrical units.

In one embodiment, the electrical system comprises a liquid sensitive unit, particularly an electrical liquid sensitive unit. The liquid sensitive unit may be arranged and configured to operate in response to or react to the exposure of the electrical system, for example of the liquid sensitive unit, to a liquid. By means of the liquid sensitive unit, the electrical system may be triggered to or caused to change its state. The change in state may be caused by the liquid sensitive unit and its exposure to liquid.

In one embodiment, the liquid sensitive unit comprises at least one liquid sensing arrangement. The liquid sensing arrangement may be arranged and configured to be exposed, e.g. directly exposed, to liquid. The liquid sensing arrangement may be arranged at a location, where it is particularly likely that liquid will enter the electrical system, e.g. close to an exterior of the system. The liquid sensing arrangement may be positioned in or along a path between an exterior of the electrical system and the electrical unit of the electrical system, e.g. an electronic unit. Thus, before the liquid reaches the electrical unit, the liquid sensing arrangement is exposed to liquid. In consequence, appropriate measures may be taken or triggered in response to the exposure. The liquid sensitive unit may be configured to react to the exposure of the liquid sensing arrangement to liquid.

In one embodiment, the electrical system is configured to change its state in response to the exposure of the at least one liquid sensing arrangement to liquid. The system may be configured to change its state from a normal state into an error state. In the normal state the electrical system may be operational. That is to say the electrical system may perform the functions it is envisaged to perform, e.g. recording or gathering dose information during the (dose delivery) operation of the drug delivery device. In the error state, the electrical system or may be fully operational or not fully operational, e.g. non-operational or operational but with one or more selectively deactivated electrical units.

The electrical system may be configured to indicate that the electrical system was exposed to liquid, for example if the electrical system is operational in the error state. The indication may be or may comprise an error code generated by the system. The indication may be transmitted, e.g. wirelessly, to another device, e.g. a PC, laptop, smart phone or a smart watch, and/or recorded in a memory of the system, for example a non-volatile memory. By way of the indication, information can be retrieved from the electrical system or provided by the electrical system that the functionality of the electrical system may have been affected by an exposure of components of the electrical system, e.g. an electronic unit thereof, to liquid. Alternatively or additionally to the error code, the indication may comprise an alarm, e.g. a visual, audible or tactile alarm, can be generated by the electrical system in response to the exposure of the liquid sensing arrangement to liquid. As it is then known that the electrical system has been exposed to liquid, appropriate measures may be taken, e.g. by questioning the correctness of dose information gathered by the electrical system by the user and/or a medical professional.

If the electrical system is not fully operational in the error state, the electrical system may be configured to switch off one or more functionalities or units of the electrical system, e.g. only temporarily, for example as long as the liquid sensitive unit provides the information about a continuing exposure to liquid, or permanently. If the electrical system is non-operational in the error state, the system may be powerless. For example, the switching from the normal state into the error state may be achieved by draining electrical power from an electrical power supply of the electrical system, for example the only power supply of the system. The power may be drained from the power supply via the liquid sensitive unit when the unit is exposed to liquid. The power supply may be a rechargeable or non-rechargeable battery. In the error state, the power supply may be powerless. That is to say, the power supply may not have enough power stored within it to operate the electrical system.

In one embodiment, the liquid sensitive unit, e.g. the at least one liquid sensing arrangement, is configured to react to the exposure to electrically conductive liquids, e.g. non-distilled water. Most of the liquids to which the delivery devices are exposed are usually electrically conductive. Especially, users of drug delivery devices may accidentally place the device in a region which is exposed to liquid or spill liquid over the device. The electrical conductivity of the liquid can be utilized to detect that the liquid sensing arrangement has been exposed to liquid and/or to switch the system to the error state. In one embodiment, the liquid sensitive unit, e.g. the at least one liquid sensing arrangement, is configured not to react to the exposure to non-conductive liquids, e.g. distilled water. In the regular use environment of drug delivery devices non-conductive liquids are extremely rare such that having the system react only to conductive liquids should cover almost all of the practically occurring liquid exposure situations.

In one embodiment, the liquid sensitive unit is configured to react to the exposure of the liquid sensing arrangement to liquid by changing an electrical characteristic of the liquid sensitive unit. The electrical characteristic may be the electrical resistance, e.g. between conductors of the liquid sensing arrangement (see further below). Exposure to liquid may lower the resistance due to electrically conductive liquid providing a contribution to the electrical conductance of the liquid sensing arrangement or liquid sensing circuit of the liquid sensitive unit. The liquid may conductively connect two conductors of the liquid sensing arrangement.

In one embodiment, the liquid sensing arrangement comprises a first surface. The liquid sensing arrangement may comprise a second surface. The respective surface may be electrically conductive. The first surface may be a surface of a first conductor. The second surface may be a surface of a second conductor. The first surface and the second surface may face in the same direction. The first surface and/or the second surface may be plane. The first surface and/or the second surface may be arranged and configured to be contacted by liquid, e.g. by a drop of liquid. In other words, the first surface and the second surface may be exposed. The first surface and the second surface may be arranged to be contacted simultaneously by liquid, e.g. by a drop of liquid, such as water. The exposure of the surface(s) or conductor(s) to the liquid may alter an electrical property or characteristic of the liquid sensitive unit or the liquid sensing arrangement, e.g. the resistance. This alteration may cause the electrical system to switch its state, e.g. from the normal state into the error state.

In one embodiment, the first surface and the second surface are electrically insulated or electrically separated from one another. The first conductor and the second conductor may, during operation of the system, be conductors of different electrical potentials or of the same potential.

In one embodiment, the system comprises a conductor carrier, e.g. a circuit board, such as a printed circuit board. The at least one liquid sensing arrangement may be provided on the conductor carrier. The first surface and the second surface may be provided and/or extend along the conductor carrier. The first conductor and/or the second conductor may be provided on the conductor carrier. For the first conductor and the second conductor, the conductor carrier may be a common conductor carrier. In other words, both conductors may be provided on the conductor carrier. The first surface and the second surface may be arranged on the same side or surface of the conductor carrier.

In one embodiment, the first surface and the second surface are arranged such that electrically conductive liquid, e.g. a drop of the liquid, can electrically conductively connected the first surface and the second surface, for example by bridging a gap between the first surface and the second surface. When the first surface and the second surface are conductively connected, so are the first conductor and the second conductor.

In one embodiment, the first surface and the second surface are arranged such that a maximum distance between the first surface and the second surface is less than or equal to one of the following values: 2 mm, 1 mm, 0.5 mm, 0.4 mm, 0.3 mm, 0.2 mm, 0.1 mm. This distance may specify the size of the gap between the first surface and the second surface. The smaller the distance the higher the sensitivity may be for drops or droplets of the liquid. Moreover, the smaller the distance, the less space is consumed by the liquid sensing arrangement, e.g. on the conductor carrier or circuit board.

In one embodiment, the electrical system comprises a system housing. The first surface and/or the second surface may be arranged in an interior of the system housing. The liquid sensitive unit, for example the entire liquid sensitive unit, may be arranged in the interior of the system housing. The system housing may be or may comprise a user interface member body of a user interface member of or for the drug delivery device. In other words, the electrical system may be or may comprise a user interface member for the drug delivery device, e.g. a knob and/or a button. The user interface member body of the user interface member may be manipulated by the user, e.g. by touching an exterior surface of the user interface member body, for a dose setting operation and/or for a dose delivery operation of the drug delivery device. The manipulation may involve movement of the user interface member, e.g. relative to a device housing of the drug delivery device and/or relative to a drug reservoir or drug reservoir retainer of the drug delivery device. The movement may involve axial movement (e.g. for dose delivery) and/or rotational movement (e.g. for dose setting) of the user interface member.

In one embodiment, the first conductive surface extends, e.g. circumferentially, along the (outer) perimeter of the second conductive surface or the second conductor, for example along the entire outer perimeter of the second surface or the second conductor and/or when seen in top view onto the first surface and/or the second surface. Between the outer perimeter of the second surface or conductor and the inner perimeter of the first surface or conductor the gap may be formed which can be bridged by liquid to trigger the liquid sensitive unit, e.g. to switch from the normal state to the error state. As the first surface may surround the second surface entirely in the circumferential direction liquid sensing can occur along the entire (outer) perimeter of the second surface.

In one embodiment, at least one of the first conductive surface and the second conductive surface defines or delimits an interior, e.g. when seen in top view onto the first surface and the second surface and/or when seen in top view onto the conductor carrier. For example, the first surface and the second surface define or delimit an interior. The second surface may be arranged in the interior defined or delimited by the first surface. The first surface and/or the second surface may be closed in the circumferential direction. The first surface and/or the second surface may be ring-like. For example, the first surface and the second surface may extend along a perimeter or edge of the conductor carrier. At least one electrical component may be arranged in the interior defined by the first surface and/or the second surface. The electrical component may be an electronic or non-electronic component. The component may be mounted on the conductor carrier. Accordingly, the liquid sensing arrangement may surround the component. Hence, exposure of the system to liquid may be detected before the liquid reaches the component. The component expediently is sensitive, e.g. will be damaged or no longer function properly, when exposed to liquid. The component may comprise any arbitrary one or any arbitrarily selected plurality of or all of the electrical units described further below or one or more components of the respective electrical unit.

In one embodiment, the first surface and/or the second surface is provided or formed by an electrically conductive coating, e.g. a metal or metal alloy coating. The coating may cover a portion of the conductor associated with the surface, i.e. the first conductor for the first surface or the second conductor for the second surface. The covered portion of the conductor may not be exposed, i.e. the covered portion of the conductor may not be contactable by liquid. For example, the coating is less easily oxidizable than the portion of the conductor below the electrically conductive coating (i.e. the covered portion of the conductor coated with the electrically conductive coating). The coating may be made of or comprise a noble metal, e.g. gold. The portion below the coating may be made of or comprise a non-noble metal, e.g. copper. The coating may form the entire exposed surface of the liquid sensing arrangement. The provision of the coating facilitates stable liquid sensing properties of the liquid sensing arrangement, even if the arrangement is exposable or exposed to an environment, which comprises oxygen, such as during storage or while it is used. Without the coating, the first surface and/or the second surface can be easily oxidized which might change the electrical characteristic of the liquid sensing arrangement and, consequently, render liquid detection or the reaction to liquid exposure less constant over time. The electrical system may have to be ready for operation for a long time, e.g. for one year or more or two years or more, such as up to three years or even longer. During that time, the liquid sensitive unit should reliably react to the exposure to liquid in a constant manner irrespective of how long the electrical system has been in use or in storage.

In one embodiment, the first surface and the second surface are separated by an insulating region. The insulating region may be an electrically insulating region. The insulating region may be formed of an electrically insulating material. The insulating region may be a region of the conductor carrier. The insulating region may be arranged between the first surface and the second surface. The insulating region may directly connect the first surface and the second surface. That is to say, when seen in top view onto the arrangement with the first surface, the insulating region and the second surface (e.g. in top view onto the conductor carrier), the insulating region may adjoin directly the first surface and the second surface. The insulating region may extend between the first surface and the second surface and, for example, along the entire (inner) perimeter of the first surface and/or along the entire (outer) perimeter of the second surface, e.g. when seen in top view onto the conductor carrier. The insulating region may be part of the liquid sensing arrangement.

In one embodiment, the surface of the conductor carrier in the region comprising the first surface, the insulating region, and the second surface is non-plane, e.g. profiled.

In one embodiment, the insulating region protrudes with respect to at least one of the first surface and the second surface. The insulating region may protrude with respect to (only) the first surface or (only) the second surface or both surfaces. Accordingly, the surface of the conductor carrier may have a profiled or structured surface in the region of the liquid sensing arrangement. A non-plane surface configuration of the liquid sensing arrangement may enhance the formation of liquid drops or the confinement of liquid drops in the region with the profiled surface. As the profiled surface is provided in the region of the liquid sensing arrangement, the liquid can be more easily kept at a particular suitable place and their presence can be used to trigger the reaction of the liquid sensitive unit.

In one embodiment, the insulating region is recessed with respect to at least one of the first surface and the second surface. The insulating region may be recessed with respect to (only) the first surface or (only) the second surface or with respect to both surfaces. This may also assist in forming a profiled surface

In one embodiment, the liquid sensitive unit comprises a plurality of liquid sensing arrangements. The liquid sensing arrangements may be separated from one another. The liquid sensing arrangements may be disposed in different regions of the electrical system, e.g. in different regions of the conductor carrier. The liquid sensitive unit may comprise a number of liquid sensing arrangements which is greater than or equal to: 2, 3, 4, 5, 6, 7, 8. The liquid sensitive unit may comprise a number of liquid sensing arrangements which is less than or equal to: 14, 13, 12, 11, 10, 9, 8. The respective liquid sensing arrangement may have a first conductive surface and/or a second conductive surface. The first conductive surfaces of different liquid sensing arrangements may be spatially separated from one another. The same may hold for the second conductive surfaces of different liquid sensing arrangements. Each liquid sensing arrangement may be configured as has been described above. A plurality of liquid sensing arrangements may be distributed over one surface of the conductor carrier, e.g. evenly and/or in a circumferential or angular direction. The circumferential or angular direction may be determined with respect to a surface normal of a main surface of the conductor carrier, e.g. through a geometric center of that surface. At least one liquid sensing arrangement may be provided on one surface of the conductor carrier (e.g. a first main surface of the conductor carrier) and at least one liquid sensing arrangement may be provided on another, for example opposite, surface of the conductor carrier (e.g. a second main surface of the conductor carrier). The first conductive surfaces of different liquid sensing arrangements may be formed by a common first conductor. That is to say, the first conductive surfaces may be directly conductively interconnected and/or of the same electrical potential, when the liquid sensitive unit is operated. Of course, it is also possible that different conductors are used for providing the respective first conductive surface. It may be more efficient to have one conductor, which provides different first conductive surfaces for different liquid sensing arrangements. The second conductive surfaces of different liquid sensing arrangements may be formed by a common second conductor. Again, separate conductors are possible for providing the second conductive surfaces of the respective liquid sensing arrangements.

In one embodiment, the liquid sensing arrangements are distributed around an electrical component, for example on the conductor carrier. The component may be an electronic or non-electronic component. Alternatively or additionally, the liquid sensing arrangements may be distributed along a perimeter of the conductor carrier. In this way, particularly sensitive components may be surrounded by a group of liquid sensing arrangements. The component, again, may be any one of the electrical components or electrical units discussed further below, e.g. as a component of one of the units described below. Also, more than one component on the conductor carrier may be surrounded by the liquid sensing arrangements, e.g. arrangements of the same group.

In one embodiment, the at least one electrical unit comprises at least one of, an arbitrarily selected plurality of or all of the following:
an electronic control unit. The electronic control unit may comprise a processor, e.g. a microcontroller or an ASIC (Application Specific Integrated Circuit). The electronic control unit may control operation of the electrical system and perform various functions of the electrical system, such as instruct the electrical system to measure relative movement of members of a dose setting and drive mechanism of the drug delivery device. The electrical system may be configured to calculate dose information, e.g. in the electronic control unit, from the results of the measured relative movement.
a communication unit. The communication unit may be an electronic unit. The communication unit may be a wireless communication unit. The communication unit may be configured to provide an interface to an external device, e.g. a PC, a laptop, smart phone or a smart watch.
a memory unit. The memory unit may be an electronic unit. The memory unit may be configured to store dose information. The memory unit may be or may comprise a non-volatile electronic memory.
a power supply. The power supply may be an electrical unit. The power supply expediently provides power to the electrical system for operating the electrical system. The power supply may be connected to the electronic control unit. The power supply may be pre-charged. The power supply may be a rechargeable or non-rechargeable battery.
a feedback unit, e.g. an optoelectronic unit; the feedback unit may be configured to provide a feedback or an alarm, e.g. a visual feedback or alarm, to the user about a status of the system, e.g. whether the system is in the normal state or in the error state. The feedback unit may comprise one or more light-emitting diodes.
a sensing unit, e.g. a motion sensing unit. The (motion) sensing unit may comprise a sensor, e.g. one or more sensors. The (respective) sensor may be an electronic sensor, e.g. an optoelectronic sensor. The (respective) sensor may provide measurements results or readings which are sensitive to the presence of liquid, e.g. at the sensor or in a detection region covered by the sensor. In other words, liquid may have a negative influence on the sensor's performance and/or the accuracy of its readings. The (respective) sensor may be an electrically and/or optically operating sensor, e.g. a radiation detector. The motion sensing unit may be configured to provide information on the size of set and/or the delivered dose based on readings taken by the sensor during operation of the system and/or the drug delivery device.

In one embodiment, the first conductive surface and/or the second conductive surface or the associated conductor is, for example directly, conductively connected to the electrical unit. The electrical unit may be part of the liquid sensitive unit. In this way, when the liquid sensing arrangement is exposed to liquid, the liquid may cause a conductive connection between the first surface and the second surface which, before the liquid exposure occurred, was not present.

The electrical unit may be the power supply. The conductive connection between the first surface and the second surface, for example, causes a short circuit which results in the power supply being discharged. When the power supply has been discharged, the electrical system may be inoperable. This provides a fail safe reaction of the electrical system to liquid exposure as due to the exposure to liquid the power is drained from the power supply and no longer available to power the electrical system. When the power has been drained entirely due to the continuous presence of liquid, the system is no longer operable. When the electrical system is inoperable, the functionality of a drug delivery device comprising the electrical system for setting and delivering doses of drug may still be available. However, dose information can no longer be gathered and/or recorded by the electrical system.

Alternatively or additionally, the electrical unit may be or may comprise the electronic control unit. The conductive connection between the first conductive surface and the second conductive surface may be detectable by the electronic control unit, e.g. by a change in electrical characteristic of a circuit comprising the first surface and the second surface as well as, for example, the electronic control unit. The change in the electrical characteristic may be detectable by and/or can be evaluated in the electronic control unit. The electrical characteristic, e.g. the resistance, may be monitored by the electronic control unit. In case the electronic control unit indicates based on the monitored characteristic that the liquid sensing arrangement has been exposed to liquid (which may mean a lower resistance detectable by the electronic control unit), the electronic control unit may react to the liquid exposure. For example, the electronic control unit may generate an according error code which may be transmitted to an external device and/or stored in the memory unit to indicate that the electrical system has been exposed to liquid. Alternatively or additionally, selected units which are potentially affected by the liquid may be deactivated or all electrical functions or units are deactivated. If the system was exposed to liquid the electrical or electronic functions, like calculating the delivered dose from data generated by the motion sensing unit, may not be performed properly. Once liquid exposure has been detected via the liquid sensitive unit, appropriate measures can be taken, e.g. by not considering the calculated dose information for a dose history or correcting the dose information. In response to the detection of the liquid exposure, the electronic control unit may instruct the feedback unit to generate a visual signal indicative for the error state of the system.

In one embodiment, the drug delivery device is a pen-type device and/or an injection device.

In one embodiment, the drug delivery device is a device of the dial extension type. That is to say the user interface member which is moved during the dose setting operation is displaced away from the housing of the drug delivery device in an amount proportional to the size of the set dose (setting a dose often is also called dialing a dose).

In one embodiment, the drug delivery device comprises a drug reservoir and/or a reservoir retainer for receiving a drug reservoir, e.g. a cartridge holder. The reservoir, e.g. a cartridge, may hold a drug. The reservoir retainer may be releasably coupled to the housing of the drug delivery device for replacing a used reservoir with a new one. Alternatively, the reservoir retainer may be permanently coupled to the housing.

In one embodiment, the drug delivery device comprises a drug delivery device unit to which the electrical system may be, for example releasably, attached. In this case, the drug delivery device unit for example has a reservoir retainer which is permanently coupled to the housing.

In one embodiment, the electrical system may be integrated into the drug delivery device, e.g. such that it cannot be removed from the device. In this case, the reservoir retainer or the reservoir is for example releasably coupled to the housing such that a new reservoir may be operatively coupled to a dose setting and drive mechanism of the drug delivery device.

In one embodiment, the drug delivery device is a carry-on device. Carry-on devices are devices which are carried by the users with them to be used when needed, e.g. in a bag. Hence, these devices are particularly prone to liquid exposure.

In one embodiment, the drug delivery device is a self-administration device. Devices of this kind are used by medically untrained persons to administer a drug.

It is noted that the features described in connection with different aspects or embodiments above or below can be combined with each other.

In a particularly advantageous embodiment, an electrical system for a drug delivery device comprises:
an electrical liquid sensitive unit, wherein the electrical liquid sensitive unit comprises at least one liquid sensing arrangement, wherein the electrical liquid sensitive unit is configured to react to the exposure of the liquid sensing arrangement to liquid, and
wherein the electrical system is configured to change its state from a normal state to an error state in response to the exposure of the at least one liquid sensing arrangement to liquid.

Further features, advantageous embodiments and expediencies of the presently disclosed subject-matter are described below in conjunction with the drawings.

### BRIEF DESCRIPTION OF THE DRAWINGS

FIG. 1 illustrates an embodiment of a drug delivery device.

FIG. 2 illustrates an embodiment of an electronic system.

FIG. 3 illustrates an embodiment of a liquid sensitive unit.

FIG. 4 illustrates another embodiment of the liquid sensitive unit.

FIGS. 5A and 5B illustrate embodiments of the liquid sensitive unit.

Identical features, identically acting features and features of the same kind may be provided with the same reference numerals in the drawings.

### DETAILED DESCRIPTION

In the following, some embodiments will be described with reference to an insulin injection device. The present disclosure is however not limited to such application and may equally well be deployed with injection devices that are configured to eject other medicaments or drug delivery devices in general, for example pen-type devices and/or injection devices. Embodiments are provided in relation to injection devices, in particular to variable dose injection devices, which record and/or track data on doses delivered therewith. These data may include the size of the selected dose and/or the size of the actually delivered dose, the time and date of administration, the duration of the administration and/or the like.

Certain embodiments in this document are illustrated with respect to an injection device where an injection button and grip (dose setting member or dose setter) are combined. Such a device is disclosed in WO 2014/033195 A1 or WO 2014/033197 A1, for example, the disclosure content of these documents being included herein in its entirety for all purposes. The injection button may provide the user interface member for initiating and/or performing a dose delivery operation of the drug delivery device. The grip or knob may provide the user interface member for initiating and/or performing a dose setting operation. These devices are of the dial extension type, i.e. their length increases during dose setting. Other injection devices with the same kinematical behavior of the dial extension and button during dose setting and dose expelling operational mode are known as, for example, the Kwikpen® device marketed by Eli Lilly and the Novopen® 4 device marketed by Novo Nordisk. An application of the general principles to these devices therefore appears straightforward and further explanations will be omitted. However, the general principles of the present disclosure are not limited to that kinematical behavior. Certain other embodiments may be conceived for application to injection devices where there are separate injection button and grip components/dose setting members (e.g. the device disclosed in WO 2004/078239 A1 the disclosure content of which is included herein in its entirety for all purposes). Thus, there may be two separate user interface members, one for the dose setting operation and one for the dose delivery operation.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing 10 end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

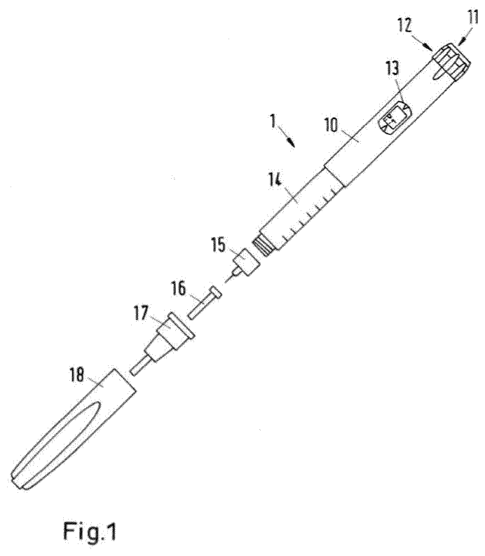

FIG. 1 is an exploded view of a medicament delivery device or drug delivery device 1. In this example, the medicament delivery device is an injection device 1, e.g. a pen-type injector, such as an injection pen. The device may be or may correspond to the one also disclosed in WO 2019/101962 A1, the disclosure of which is herewith explicitly incorporated by reference into the present application in its entirety, especially as regards the dose setting and drive mechanism and the electronic functionalities disclosed therein.

The injection device 1 of FIG. 1 is an injection pen that comprises a housing 10 and contains a container 14, e.g. an insulin container, or a receptacle for a container 14, e.g. with the container in the receptacle or insertable into the receptacle. The container may contain a drug. The container may comprise drug in an amount sufficient for setting and delivering a plurality of doses of the drug.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N-(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F (ab) and F (ab′) 2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′) 2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

A needle 15 can be affixed to the container or the receptacle. The container may be a cartridge and the receptacle may be a cartridge holder. The needle is protected by an inner needle cap 16 and an outer needle cap 17 and/or another cap 18. An insulin dose to be ejected from injection device 1 can be set, programmed, or ‘dialled in’ by turning a dosage knob 12, and a currently programmed or set dose is then displayed via dosage window 13, for instance in multiples of units. The indicia displayed in the window may be provided on a number sleeve or dial sleeve. For example, where the injection device 1 is configured to administer human insulin, the dosage may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dosage window 13 in FIG. 1. The dosage window 13 may be in the form of an aperture in the housing 10, which permits a user to view a limited portion of a dial sleeve 20 or number sleeve that is configured to move when the dosage knob 12 is turned, to provide a visual indication of a currently set dose. The dosage knob 12 is rotated on a helical path with respect to the housing 10 when setting a dose.

In this example, the dosage knob 12 includes one or more formations to facilitate attachment of a data collection device or electronic system. Alternatively, the electronic system may be integrated into the device.

The injection device 1 may be configured so that turning the dosage knob 12 causes a mechanical click sound to provide acoustic feedback to a user. In this embodiment, the dosage knob or dose button 12 also acts as an injection button 11. When needle 15 is stuck into a skin portion of a patient, and then dosage knob 12/injection button 11 is pushed in an axial direction, the insulin dose displayed in display window 13 will be ejected from injection device 1. When the needle 15 of injection device 1 remains for a certain time in the skin portion after the dosage knob 12 is pushed, the dose is injected into the patient's body. Ejection of the dose may also cause a mechanical click sound, which may be different from the sounds produced when rotating the dosage knob 12 during dialing of the dose.

In this embodiment, during delivery of the insulin dose, the dosage knob 12 is returned to its initial position in an axial movement, without rotation, while the dial sleeve 20 is rotated to return to its initial position, e.g. to display a dose of zero units. As noted already, the disclosure is not restricted to insulin but should encompass all drugs in the drug container 14, especially liquid drugs or drug formulations.

Injection device 1 may be used for several injection processes until either the insulin container 14 is empty or the expiration date of the medicament in the injection device 1 (e.g. 28 days after the first use) is reached. In the case of a reusable device, it is possible to replace the insulin container. In case an electronic system is integrated into the device, the device is for example reusable. If the electronic system is configured to be an add-on, which can be connected to a drug delivery device unit, the unit may be a reusable unit, e.g. with or for a replaceable container, or a disposable unit.

Furthermore, before using injection device 1 for the first time, it may be necessary to perform a so-called “prime shot” to remove air from container 14 and needle 15, for instance by selecting two units of insulin and pressing dosage knob 12 while holding injection device 1 with the needle 15 upwards.

As explained above, the dosage knob 12 in the present embodiment also functions as an injection button 11 so that the same component or user interface member is manipulated for dialing/setting the dose and for dispensing/delivering the set dose.

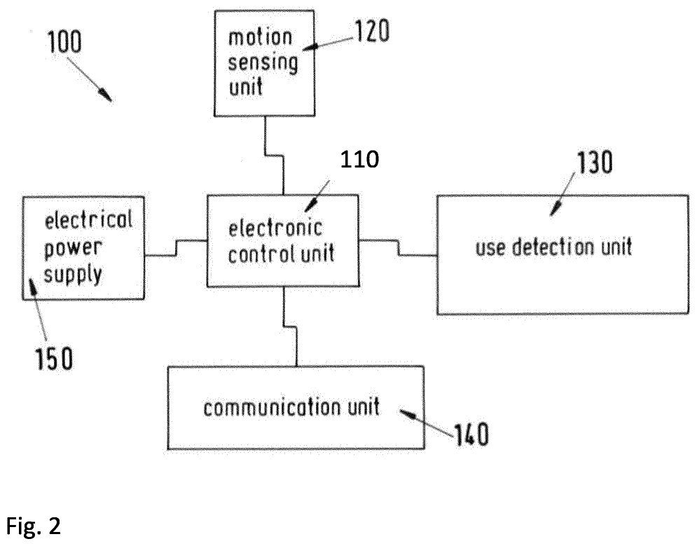

A schematic representation of an embodiment of the electronic system is shown in FIG. 2. The system 100 may comprise a dose setting and drive mechanism which may be part of an injection device 1 as depicted in FIG. 1 or is configured to be mechanically connected to such a device unit having such a mechanism as an add-on module. The module may provide electronic functionality to a device having an electronic free drug delivery device unit (hence, all electrical or electronic components may be contained in the add-on module). The exterior of the system may be defined by the button 11 and/or the knob 12 which may form a user interface member body.

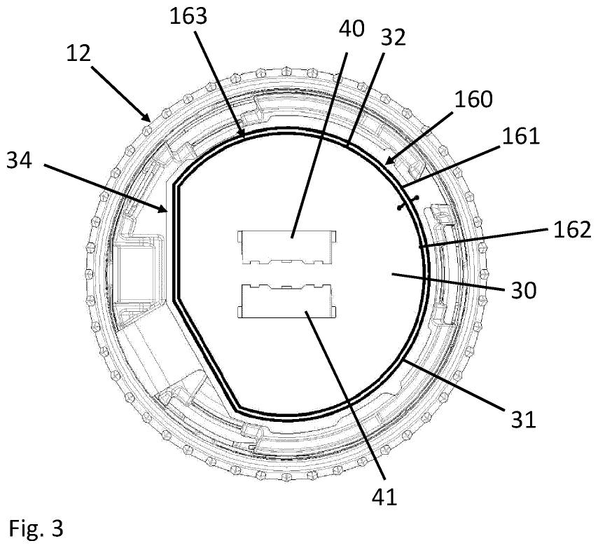

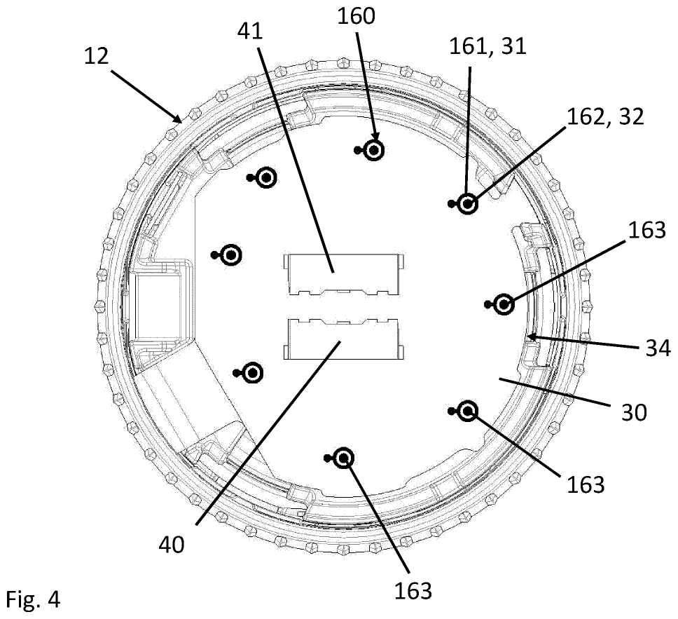

The electronic system 100 comprises an electronic control unit 110. The control unit may comprise a controller. Specifically, the control unit may comprise a processor arrangement, e.g. a microcontroller or an ASIC. Also, the control unit 110 may comprise one, or a plurality of memory units, such as a program memory and a main memory. The control unit 110 is expediently designed to control operation of the electronic system 100. The control unit 110 may communicate via wired interfaces or wireless interfaces with further units of the system 100. It may transmit signals containing commands and/or data to the units and/or receive signals and/or data from the respective unit. The connections between the units and the electronic control unit are symbolized by the lines in FIG. 2. However, there may also be connections between the units which are not illustrated explicitly. The control unit 110 may be arranged on a conductor carrier, e.g. a (printed) circuit board, (see reference “30” in FIGS. 3 and 4). The other unit(s) of the system may comprise one or more components which are arranged on the conductor carrier as well.

Electronic system 100 may further comprise a motion sensing unit 120. The motion sensing unit 120 may comprise one or a plurality of sensors. In case optoelectronic sensors which detect electromagnetic radiation, such as IR sensors, are used, the motion sensing unit may additionally comprise a radiation emitter which emits the radiation to be detected by the sensor. However, it should be noted that other sensor systems, e.g. magnetic sensors could be employed as well. A motion sensing unit which has an electrically operated sensor and an electrically operated source for stimulating the sensor—such as a radiation emitter and an associated sensor—the power consumption may be particularly high and, hence, power management may have particular impact as the capacity of a power supply may be limited. Each sensor may have an associated radiation emitter. Motion sensing unit 120 may be designed to detect and for example measure relative movement of two movable members of the dose setting and drive mechanism of or for the drug delivery device during a dose setting operation and/or during a dose delivery operation. For example, the motion sensing unit may measure or detect relative rotational movement of two movable members of the dose setting and drive mechanism with respect to one another, e.g. the dose dial member and the user interface member or the button. Based on movement data received from or calculated from the signals of the unit 120, the dose information may be calculated, e.g. by the control unit 110. The dose information may be data on the size of the delivered dose.

Electronic system 100 may further comprise a use detection unit 130. The use detection unit may be associated with the user interface member, e.g. button 11, such that manipulation of the member for setting and/or delivering a dose thereof may be detected. When the manipulation is detected, the use detection unit generates or triggers generation of a use signal. The use signal can be transmitted to the electronic control unit 110. The electronic control unit may, in response to the signal, issue a command or signal to one of, an arbitrarily selected plurality of, or all of the other electrically operated units of the system. For example, the control unit may cause that the respective unit is switched from a first state, e.g. a sleeping state or idle state with a lower power consumption or an off state with no power consumption, to a second state with an increased power consumption, e.g. a state where respective unit is operational. The switching may be done by an according switching command or signal issued by the electronic control unit to the respective unit. In response to the use signal all units may be switched to the second state or just selected units. If only selected units are switched to the second state with higher power consumption, it is expedient that these units are intended to be used during the operation which is intended to be commenced by the user or which has been commenced when the manipulation of the user interface member has been detected. The motion sensing and/or communication unit may have particularly high power consumption. Hence, switching one (e.g. motion sensing unit or communication unit) or both of these units to a state of higher power consumption, e.g. a state in which they are operational or can be operated as intended, in response to the use signal is particularly advantageous.

The electronic system 100 may further comprise a communication unit 140, e.g. an RF, WIFi and/or Bluetooth unit. The communication unit may be provided as a communication interface between the system or the drug delivery device and the exterior, such as other electronic devices, e.g. mobile phones, personal computers, laptops, smart watches and so on. For example, dose information may be transmitted by the communication unit to the external device. The dose information may be used for a dose log or dose history established in the external device. The communication unit may be provided for wireless or wired communication.

The electronic system may further comprise an electrical power supply 150, such as a rechargeable or non-rechargeable battery. The power supply 150 may provide electrical power to the respective units of the electronic system.

Although not explicitly depicted, the electronic system may comprise a, for example permanent and/or non-volatile, storage or memory unit, which may store data related to the operation of the drug delivery device such as dose data or history data, for example.

The system 100 may further comprise a feedback unit, e.g. an optical feedback unit (not explicitly shown in FIG. 2). The feedback unit may comprise one or more light-emitting diodes. The light-emitting diodes may be arranged on the conductor carrier (see the description below).

Drug delivery devices are often carried along by their users and/or stored for longer periods of time. During storage or while they are carried on or even when they are used, there is a risk that liquid, e.g. water, enters the device. Liquid entering a purely mechanically operated device is not that critical, as mechanical drug delivery devices are usually robust with respect to liquid exposure. If electrically powered components or units are used for drug delivery devices, however, the situation is fundamentally different. For example, if systems with one or more than one electrically operated units are employed, there is an increased risk that the system does not operate properly anymore when having been exposed to liquid.

Components might be damaged or the functionality of electronic elements of the device may be negatively impacted. For example, measurements conducted via the motion sensing unit, which can operate optically, might be distorted or the communication unit might be rendered inoperable.

Further, the system may be required to interface with mechanical elements of the drug delivery device. Such interfaces, may advantageously be via mechanical contact, which could allow mechanical switches to change state during use of the device. The switch(es) may be part of the use detection unit described further above. Switch state change can be implemented at low cost, and can enable very low power consumption when the device is not in use. Alternatively, remote (non-contact) sensing may require higher power consumption, to continuously monitor or regularly monitor the state of the device whilst not in use, via a sensing system which requires energy to obtain a reading. Mechanical contact may then require apertures in the electrical system, e.g. close to components of the dose setting and drive mechanism of the drug delivery device, e.g. a drive member. These apertures could be sealed, but this is likely to increase size, complexity, cost and might inhibit free movement of elements of the switch. Hence a preferred solution for detecting when the device is in use may necessitate apertures in the housing of the system.

Functional requirements of the device may require robust connections between components of the system and the dose setting and drive mechanism of the device, where the mechanism may operate without electricity, and/or between internal components of the system. Achieving robust connections often requires apertures in the components to form sharp or undercut clip surfaces, without adding additional complexity to manufacturing tools. Additional components or processes could be added to seal each of these apertures, but these additional components would add cost, sized and complexity to the design, and may themselves introduce additional risk. Hence a preferred solution for robust mechanical connections may necessitate apertures in the electrical system.

Just as detecting whether the device is intended to be used (e.g. via the use detection unit 130), capturing and/or recording the dialling and/or dispensing or delivering of a dose (e.g. via the motion sensing unit 120) may require additional sensors. Capturing and/or recording doses can be achieved via a remote sensing system, which would only need to be active when a state change sensor or switch (for example of the use detection unit) is triggered. A number of sensing systems can be considered, including but not limited to, optical, magnetic, acceleration and capacitive. For a low cost, compact, reliable device, it may be preferable to utilise a sensor which gives a consistent output in all operating conditions and is not influenced by external factors. It may also be preferable to utilise a sensor technology which does not add complexity to the mechanical portion of the device, which is typically manufactured from injection moulded plastic components. Optical sensors, which detect the colour or reflectivity change of elements of the dose setting and drive mechanism of the device may be a particular low cost, reliable solution. It may be preferable to position the optical sensors close to elements of the dose setting and drive mechanism, and to ensure that no obstruction occurs between the optical sensor and a target element of the dose setting and drive mechanism, e.g. the drive member or the dose dial member or number sleeve. Such a solution may also lead to the need for apertures within the system.

Thus, for a variety of reasons, apertures may be needed within the electrical system (although no apertures would be ideal with respect to the protection against liquid ingress), where each aperture presents a potential path via which liquid could enter the electrical system. Therefore, it is desirable to include a functionality into electrical systems for drug delivery devices or devices comprising such systems, the functionality being suitable to detect liquid ingress into the system and/or to mitigate the effects thereof. The liquid, to which the system might be exposed most likely is (non-distilled) water, e.g. coming from the environment. Also, most of the liquids which the system is likely exposed to are conductive liquids.

Below, some concepts and units are proposed to detect and/or mitigate the effect of liquid ingress into an electrical system. The electrical system can comprise the electronic system disclosed further above. The electrical system may be integrated in a drug delivery device or be configured to attach to a drug delivery device unit to form a drug delivery device. In the latter case, the system may have interface features, e.g. snap features to attach to the drug delivery device unit. Particularly, liquid exposure may have significant negative effects in a pen injection device containing a system for determining, recording, and/or communicating data on typical usage of the device, e.g. the size of a delivered or set dose. The device can be intended to be used in everyday settings by patients (rather than clinical or well-controlled environments). As such, it is likely and potentially unavoidable that the device may be exposed to water (or other liquids) at some point during its life. It is therefore advantageous to provide means to mitigate the risk of this exposure, or to detect if exposure has taken place such that the functionality of the device is not compromised.

The present disclosure proposes an electrical system which has an electrical liquid sensitive unit. The liquid sensitive unit may be sensitive to and/or react to electrically conductive liquids, e.g. only to electrically conductive liquids. Specifically, the liquid sensitive unit may use the electrical conductivity of the liquid as an indicator or trigger that liquid is present. By way of the unit it may be detected whether exposure to liquid has taken place and/or the unit may be designed such that the effect of the liquid is mitigated. The system may be configured such that, in case liquid exposure is detected, an error code is being generated and/or the electronic control unit instructs one or more units of the electronic system to power down and/or send data to an external device different from the drug delivery device indicating that the system has been exposed to liquid. The effect of liquid exposure may be mitigated by deactivating one or more units which have been exposed to liquid such as by the electronic control unit 110, which may send an according command to the unit in response to the detection that the liquid sensing arrangement has been exposed to liquid.

FIG. 3 schematically illustrates one embodiment of a liquid sensitive unit 160. FIG. 3 shows the user interface member or dosage knob 12 on the basis of a sectional view. The view in FIG. 3 is a view from the proximal end of the electrical system, that is to say from the side of the injection button 11 (see FIG. 1) or the proximal end of the electrical system, onto the interior of the user interface member/dosage knob 12. In the interior of the user interface member a conductor carrier 30 is arranged. The conductor carrier 30 for example is a printed circuit board having different conductor tracks. One or more electrical or electronic units of the system may be arranged on and be electrically contact connected or mounted on the conductor carrier 30. In the depicted embodiment, two components 40 and 41 are shown. However, any other number of components may be arranged on the conductor carrier 30, e.g. one component or more than two components. The components 40 and 41 may be light-emitting diodes configured to provide a visual indication, by the emitted light, to the user about a state of the system or the device, e.g. whether the communication unit is active and/or whether the motion sensing unit is active currently. The components may belong to the feedback unit described above. In other embodiments, the components 40 and 41 may comprise the electronic control unit 110, the motion sensing unit 120 (or at least one sensor thereof), one or more sensors (e.g. sensors not necessarily belonging to the motion sensing unit), and/or the communication unit 140, for example.

The liquid sensitive unit 160 comprises a liquid sensing arrangement 163. The liquid sensing arrangement 163 comprises a first electrically conductive surface 161 and a second electrically conductive surface 162. The respective conductive surface is accessible on the surface of the conductor carrier 30 on which the component 40 of 41 is provided. The first conductive surface 161 may be formed by an exposed portion of a first conductor or conductor track 31 of the conductor carrier 30. The second conductive surface 161 may be formed by an exposed portion of a second conductor or conductor track 32 of the conductor carrier 30. The first and second conductors or the first and second surfaces respectively are electrically separated from each other. On the main surface of the conductor carrier 30 on which the conductive surfaces are accessible there is no electrical interconnection between these elements. The conductors 31, 32 may be conductors of different electrical potentials, e.g. of opposite signs or polarity during operation of the electrical system. Alternatively, the conductors may be of the same electrical potential during operation of the electrical system.

The first surface 161 surrounds the second surface 162 entirely as seen in top view onto the main surface of the conductor carrier 30 (as is shown in FIG. 3). To put it differently, the second surface 162 is arranged within the interior defined by the first surface 161 as seen in top view onto the main surface of the conductor carrier 30. The respective component 40, 41 is also arranged in the interior defined by the first surface. The first surface may circumferentially enclose the component(s) 40, 41 as seen in top view onto the conductor carrier. The second surface 162 defines an interior and/or surrounds the component(s) entirely in the circumferential direction, just as the first surface 161.

The liquid sensing arrangement 163 (formed by the surfaces 161 and 162) extends ring-like around the component(s). Thus, a liquid sensing ring may delimit the interior of the liquid sensing arrangement from the exterior. The respective conductive surface 161, 162 of the liquid sensing arrangement 163 extends along a perimeter or edge 34 of the conductor carrier 30. The liquid sensing arrangement is expediently located close to the edge or perimeter 34 of the conductor carrier 30 and extends along the edge or perimeter. Particularly, the liquid sensing arrangement may be arranged with a distance of less than or equal to 0.5 cm from the edge or perimeter of the conductor carrier, for example along the entire extension of the liquid sensing arrangement on the main surface. Given the fact that liquid ingress will usually occur from the exterior of the electrical system, the liquid will likely reach the edge region of the conductor carrier before it reaches the (respective) component 40, 41. The exposure to liquid will likely occur at the liquid sensing arrangement 163 before an electrical component of the electrical system is exposed to liquid. Hence, appropriate measures can be taken timely once the liquid sensing arrangement has been exposed to liquid.

The first and second conductive surfaces 161, 162 of the liquid sensing arrangement 163 are arranged such that conductive liquid, e.g. a drop of liquid, can bridge the space or gap between the conductive surfaces 161 and 162. This will change an electrical characteristic of the liquid sensitive unit 160, e.g. the electrical resistance, between the conductors 31, 32 or conductive surfaces 161, 162. The change in electrical characteristic can trigger a reaction of the electrical system to the liquid exposure.

In one embodiment, the liquid sensing arrangement 163 may be operatively coupled, e.g. conductively connected to, the electronic control unit 110 or a dedicated liquid sensitive unit control unit. The conductors 31 and 32 may be connected to, e.g. different ports, of the control unit. The control unit may detect the exposure to liquid via the change in electrical characteristic of or at the liquid sensing arrangement. For example, the reduction in electrical resistance or the increase in conductance between the conductors or conductive surfaces is indicative for exposure of the liquid sensing arrangement to liquid. The change in resistance may be detected by a voltage or current change via the control unit. When exposure to liquid has been detected, the system is expediently configured to react to the exposure, e.g. by switching the system from a normal state to an error state. The reactions may be controlled by the electronic control unit or the dedicated control unit, for example by issuing associated instructions. The reactions can include any one or any combination of the following, for example:
generating an error code. The error code can be stored in a memory of the system and/or transmitted to an external device, e.g. a smartphone or smart watch. In this way, the software which may gather or transmit data via one or more units of the system may be aware that liquid exposure has occurred.
deactivating one or more electrical, e.g. electronic, components or units of the electrical system, e.g. comprising at least one component to which the liquid sensing arrangement indicating the exposure to liquid is assigned or which is particularly sensitive to liquid exposure, e.g. the motion sensing unit. Only some units may be deactivated or the electrical system may be deactivated in its entirety.
providing a feedback of the system to a user, e.g. a visual feedback via the feedback unit, like light of a particular color or in a predetermined flashing pattern. The feedback may be indicative for the exposure to liquid.

In case the liquid exposure is no longer present, e.g. when the liquid has dried, the system can be switched from the error state back to its normal state, e.g. by the electronic control unit, or remain in the error state.

In an alternative embodiment, i.e. alternative to the connection to the control unit, the liquid sensing arrangement may be, for example directly, conductively connected to the power source. The first surface 161 or first conductor 31 can be connected to a first terminal of the power supply. The second surface 162 or second conductor 32 can be conductively connected to a second terminal of the power supply. The first and second terminals expediently are terminals of opposite polarity. Hence, the exposure to liquid causes a short circuit of the power supply and the power supply is emptied quickly. The power supply is expediently the power supply for all electrically operated units of the system or at least the ones which should no longer operate when the system was exposed to liquid, e.g. the motion sensing unit 120 or the communication unit 140. Thus, the system can be rendered powerless when exposed to liquid. This provides a fail-safe condition of the electrical system, as it can no longer operate. Thus, incorrect operation due to liquid ingress, e.g. wrongly determined dose sizes, can be avoided. However, as opposed to the connection of the liquid sensing arrangement to a control unit, in this embodiment the electrical system or the respective unit is rendered inoperable irreversibly. That is, of course, unless additional power is supplied to the power supply, e.g. by recharging the battery.

FIG. 4 illustrates another embodiment of the liquid sensitive unit 160. In general, the functionalities correspond to the one disclosed above in connection with FIG. 3. Hence, the following focuses on the differences. The most prominent difference is that the liquid sensitive unit 160 comprises a plurality of liquid sensing arrangements 163. The arrangements 163 are distributed along the perimeter or edge 34 of the conductor carrier 30 and/or around the component 41 or 40 which may be arranged close to the center of the conductor carrier 30. Each liquid sensing arrangement 163 comprises a first conductive surface 161 and a second conductive surface 162. Again, the first conductive surface 161 surrounds the second surface 162. The second conductive surface 162 may be continuous without defining an interior or also ring-like. The first surface 161 in the depicted embodiment follows a circular line, where other configurations are possible. As opposed to the configuration of FIG. 3, this configuration may facilitate that a liquid drop is confined or constrained to the liquid sensing arrangement 163. The first conductive surfaces 161 of different liquid sensing arrangements can be directly conductively connected, e.g. in the interior of the conductor carrier 30, and/or be formed by the same first conductor 31. Alternatively or additionally, the second conductive surfaces 162 of different liquid sensing arrangements 163 can be directly conductively connected, e.g. in the interior of the conductor carrier 30, and/or be formed by the same second conductor 32. The liquid sensing arrangements 163 may be dimensioned to accommodate a liquid drop, expediently one that can bridge the gap between the conductive surfaces 161 and 162. For example, the maximum distance between diametrically opposite points on the outer edge of the first conductive surface or the first conductor of the respective liquid sensing arrangement 163 may be less than or equal to one of the following values: 3 mm, 2 mm, 1 mm, 0.9 mm, 0.8 mm, 0.7 mm, 0.6 mm, 0.5 mm, 0.4 mm, 0.3 mm, 0.1 mm, 0.05 mm. In other words, the (maximum) diameter of the respective liquid sensing arrangement 163 may be less than or equal to the values specified above.

In the following, features applying for all of the liquid sensitive units described herein are disclosed, especially the ones shown in FIGS. 3 and 4.

In the respective liquid sensing arrangement, the distance between the adjacent conductive surfaces of one liquid sensing arrangement is for example less than or equal to one of the following values: 2 mm, 1 mm, 0.5 mm, 0.4 mm, 0.3 mm, 0.2 mm, 0.1 mm. The distance may be constant along the respective liquid sensing arrangement and/or equal over all liquid sensing arrangement in case a plurality of the arrangements is provided.

The width (or radial dimension) of the respective conductive surface or conductor portion as seen perpendicular to its direction of extension on the conductor carrier may be less than or equal to one of the following: 1 mm, 0.5 mm, 0.4 mm, 0.3 mm, 0.2 mm, 0.1 mm.

The dimensions given above may be particularly suitable to hold liquid, e.g. water, at the respective liquid sensing arrangement.

At least one liquid sensing arrangement 163 of the liquid sensitive unit 160 may be provided on opposite main surfaces of the conductor carrier 30. Particularly, opposite main surfaces of the conductor carrier 30 may be provided with liquid sensing arrangements 163, e.g. arrangements as shown in FIG. 3 and/or FIG. 4. In this way, detection of or reaction to liquid exposure is less prone to the orientation of the system or the device as both opposite main surfaces of the conductor carrier may be provided with liquid sensitivity.

The respective conductive surface 161 or 162 may be formed by a coating, e.g. a gold coating. The portion of the conductor 31, 32 below the conductive surface coating may be of a different conductive material, e.g. copper. Having a different material on the exposed surface, e.g. a non-easily oxidizable material such as gold, of the associated conductor may improve the homogeneity of liquid sensing over time.

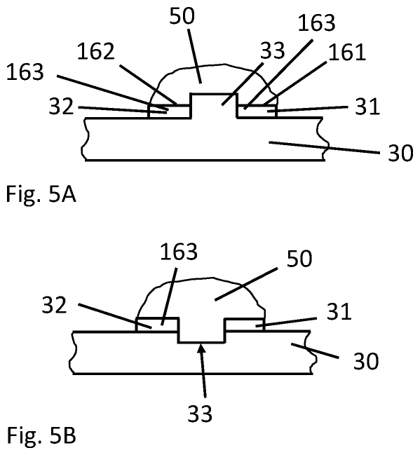

FIGS. 5A and 5B illustrate embodiments of the liquid sensing arrangement 163, e.g. the one previously described. FIGS. 5A and 5B show sectional views through the conductor carrier 30. Between the first and second conductive surfaces 161 and 162, an insulating portion 33 of the conductor carrier is provided as seen in top view onto the conductor carrier 30. In FIG. 5A, the insulating portion 33 is elevated relative to the conductors 31 and 32. In FIG. 5B the portion 33 is recessed relative to the conductors 31, 32. It is also possible that the portion 33 is elevated or recessed only with respect to one of the surfaces 161, 162 or recessed relative to one of the surfaces 161 and 162 and elevated relative to the other one. The profiled region of the main surface of the conductor carrier 30 which is formed in this way may facilitate that liquid drops like drop 50 may be more easily confined to the liquid sensing arrangement 163.

The liquid sensitive unit 160 may be switched from a first or non-operational state to a second or operational state in response to the use detection unit generating a (use) signal. Hence, the liquid sensitive unit may be operational only when the system is expected to perform a system operation such as dose information determination and/or transmission because a dose setting or delivery operation is to be expected. Alternatively, the liquid sensitive unit may be operational also when no upcoming operation is expected.

With the presently proposed concepts, upon a liquid ingress into the electrical system, drug delivery devices comprising the system may enter into a “fail safe” mode rather than that the device carries on attempting to function with the risk of dose record errors. Therefore, if liquid ingress is detected, for example a “fail safe” mode can be produced or an appropriate permanent error code could be generated.

As explained above, the detection of liquid ingress could be made with the use of exposed (copper or coated copper) tracks on the conductor carrier. Typical types of fluid that could enter into the system during lifetime are likely to have sufficient conductivity to be able to be detected by the electronics and software of the system. The proposed concepts relate to the addition of exposed (copper) tracks in regions of the conductor carrier (PCB) close to where fluid or liquid could enter the device. The optional (gold) plating or coating has the effect of maintaining conductivity over lifetime such that the effectiveness at detecting water will not change over time.

The disclosure is not limited by the description in conjunction with the exemplary embodiments. Rather, the disclosure comprises any new feature as well as any combination of features, particularly including any combination of features in the patent claims, even if said feature or said combination per se is not explicitly stated in the patent claims or exemplary embodiments.

### REFERENCE NUMERALS

1 device
10 housing
11 injection button
12 dosage knob
13 dosage window
14 container/container receptacle
15 needle
16 inner needle cap
17 outer needle cap
18 cap
20 dial sleeve
30 conductor carrier
31 first conductor
32 second conductor
33 portion
34 edge/perimeter
40 component
41 component
50 liquid drop
100 system
110 electronic control unit
120 motion sensing unit
130 use detection unit
140 communication unit
150 power supply
160 liquid sensitive unit
161 first conductive surface
162 second conductive surface
163 liquid sensing arrangement

1. An electrical system for a drug delivery device, the electrical system comprising:
an electrical liquid sensitive unit, wherein the electrical liquid sensitive unit comprises at least one liquid sensing arrangement, wherein the electrical liquid sensitive unit is configured to react to the exposure of the liquid sensing arrangement to liquid, and wherein the electrical system is configured to change its state from a normal state to an error state in response to the exposure of the at least one liquid sensing arrangement to liquid.

2. The electrical system of claim 1, wherein, in the error state, the electrical system is configured to indicate that the electrical system was exposed to liquid or, wherein, in the error state, the electrical system is powerless.

3. The electrical system of claim 1, wherein the liquid sensitive unit is configured to react to the exposure of the liquid sensing arrangement to liquid by changing an electrical characteristic of the liquid sensitive unit.

4. The electrical system of claim 1, wherein the electrical system comprises a user interface member for a drug delivery device comprising a knob and/or a button, wherein the electrical liquid sensitive unit is arranged within an interior of a user interface member body of the user interface member, wherein the user interface member body is configured to be manipulated by the user to effectuate a dose setting operation and/or a dose delivery operation of the drug delivery device by touching an exterior surface of the user interface member body.

5. The electrical system of claim 1, wherein the liquid sensing arrangement comprises a first surface and a second surface, wherein a first conductor has the first surface and a second conductor has the second surface, and wherein both surfaces are contactable by liquid.

6. The electrical system of claim 5, wherein the first surface and the second surface are arranged such that electrically conductive liquid, e.g. a drop of liquid, can conductively connect the first conductor and the second conductor by bridging a gap between the first surface and the second surface.

7. The electrical system of claim 5 or 6, wherein the first surface extends along the entire outer perimeter of the second surface.

8. The electrical system of claim 5, wherein the second surface defines an interior, an electronic component being arranged in the interior.

9. The electrical system of claim 5, wherein the first surface and/or the second surface is provided by an electrically conductive coating, the coating being less easily oxidizable than a portion of the conductor below the electrically conductive coating, where the portion is associated with the same conductor as the coating.

10. The electrical system of claim 5, wherein the first surface and the second surface are arranged on a conductor carrier.

11. The electrical system of claim 10, wherein the first surface and the second surface are separated by an insulating region, and wherein the insulating region protrudes with respect to at least one of the first surface and the second surface and/or wherein the insulating region is recessed with respect to at least one of the first surface and the second surface.

12. The electrical system of claim 10, wherein the electrical system comprises a plurality of liquid sensing arrangements, each having a first surface and a second surface, and wherein the liquid sensing arrangements are distributed around an electronic component on the conductor carrier and/or along a perimeter of the conductor carrier.

13. The electrical system of claim 5, wherein the first surface and the second surface are directly conductively connected to a power supply of the electrical system, such that exposure of the liquid sensing arrangement to liquid causes a short circuit between the first conductor and the second conductor and a discharge of the power supply.

14. The electrical system of claim 5, wherein the first surface and the second surface are directly conductively connected to an electrical unit of the electrical system, wherein the electrical unit is an electronic control unit.

15. (canceled)

16. The electrical system of claim 5, wherein the first surface and/or the second surface are closed in the circumferential direction.

17. The electrical system of claim 8, wherein the components and are light-emitting diodes configured to provide a visual indication, by the emitted light, to the user about a state of the system or the drug delivery device.

18. The electrical system of claim 11, wherein the insulating region extends between the first surface and the second surface and along the entire perimeter of the first surface and/or along the entire perimeter of the second surface.

19. (canceled)

20. (canceled)

21. A drug delivery device comprising an electrical system, the electrical system comprising an electrical liquid sensitive unit comprising at least one liquid sensing arrangement,
wherein the electrical liquid sensitive unit is configured to react to the exposure of the liquid sensing arrangement to liquid, and
wherein the electrical system is configured to change its state from a normal state to an error state in response to the exposure of the at least one liquid sensing arrangement to liquid.

22. The drug delivery device according to claim 21 configured to retain a drug container with a drug or comprising a drug container with a drug, and optionally wherein the drug delivery device is a pen-type injection device of the dial extension type.

23. A method for dispensing a drug with a drug delivery device comprising an electrical system, the electrical system comprising:
an electrical liquid sensitive unit, wherein the electrical liquid sensitive unit comprises at least one liquid sensing arrangement, wherein the electrical liquid sensitive unit is configured to react to the exposure of the liquid sensing arrangement to liquid, wherein the electrical system is configured to change its state from a normal state to an error state in response to the exposure of the at least one liquid sensing arrangement to liquid, and wherein the drug delivery device is configured to retain a drug container with a drug or comprising a drug container with a drug.
